(19) United States
(12) Patent Application Publication
(10) Pub. No.: US 2022/0395655 A1
(43) Pub. Date: Dec. 15, 2022

(54) PORTABLE MICROPROCESSOR-CONTROLLED PERISTALTIC SUCTION APPARATUS

(71) Applicant: DRW Medical LLC, Aston, PA (US)

(72) Inventors: Robert Landis, Mountainside, NJ (US); Daniel W. Tatum, Brookhaven, PA (US)

(21) Appl. No.: 17/826,610

(22) Filed: May 27, 2022

Related U.S. Application Data
(60) Provisional application No. 63/210,648, filed on Jun. 15, 2021.

(51) Int. Cl. A61M 16/04; A61M 1/00; A61M 16/08

(52) U.S. Cl. CPC A61M 16/0463; A61M 1/80; A61M 16/0833; A61M 1/73; A61M 1/743; A61M 2205/7509; A61M 2205/3344; A61M 2205/7518; A61M 2205/7536

(57) ABSTRACT

A portable suction pump for aspirating secretions from a patient's artificial airway includes a peristaltic pump head, a suction tube extending from a patient through the peristaltic pump head, the suction tube having a tee-fitting conduit, and a collection container having a plurality of ports for collecting patient media. A clinician has an ability to increase or decrease vacuum levels without increasing or decreasing flowrate and/or an ability to increase or decrease the flow rate without increasing or decreasing the vacuum levels. The tee-fitting conduit extends to a vacuum sensor port connecting to a vacuum sensor in communication with a processor, the processor controlling a proportional valve employed to regulate the vacuum levels.

### RELATED APPLICATION INFORMATION

This application claims priority to Provisional Application No. 63/210,648 filed on Jun. 15, 2021, incorporated herein by reference herein in its entirety.

### BACKGROUND

The present invention relates generally to medical devices, and more specifically, to a portable microprocessor-controlled peristaltic suction apparatus.

Suction devices are used for clearing sputum from patients artificial airways and for evacuating secretions from the mouth, glottic, and sub-glottic areas, using suction devices that utilize an air/vacuum/suction pump to generate vacuum inside a rigid upright container where the liquid is captured and settled to the bottom while the air is pumped into the atmosphere. This air/vacuum/suction pump method using a rigid-walled container for generating and storing vacuum has been the standard since World War II and even before that. Every hospital bedside in America and abroad uses this type of suction setup. The setup generally works. However, there are collateral damages, described and published in medical journals related to the adverse effects to the patient during suction procedures, more specifically when suctioning secretions out of patient's breathing tubes.

Most patients with an endotracheal tube or tracheotomy tube in place for breathing require the tube to be cleared of secretions. The is accomplished by standard suction procedures. The American Association for Respiratory Care (AARC) recently reviewed the “Best Practices for ET Tube Suctioning.” Upon review of the “Best Practices,” it is clear that the “Best Practices” are designed to mitigate collateral damages that occur during suction procedures. Nevertheless, there is not one mention in the review that the hospital wall suction system should be evaluated. The present inventors have used hospital wall suction for many years to clear patient's breathings tubes, with firsthand knowledge of how it can cause discomfort, hypoxia, heart arrhythmias, tissue damage, etc., and, thus, understand that there is room for improvement.

In order to extract secretions from a patient's ET Tube, sufficient vacuum in the rigid container is needed to get thick liquids through the catheters. However, once the fluids are cleared, there is a free-flow of air into the vacuum filled container. That free-flow is air from the patient's lungs. For example, the free-flow of air through a 14FR suction catheter at 90 mmHg negative pressure is 24 L/min. The present inventors believe this is a contributing factor to the adverse events related to ET Tube suctioning. When the vacuum level is increased to 150 mmHg, according to the “Best Practices,” the free flow increases, proportionally with the selected vacuum level. The higher the vacuum, the higher the free-flow, thus drawing air out of the patient's lungs and leading to adverse effects.

### SUMMARY

In accordance with an embodiment, a portable suction pump for aspirating secretions from a patient's artificial airway is provided. The portable suction pump includes a peristaltic pump head, a suction tube extending from the patient through the peristaltic pump head, the suction tube having a tee-fitting conduit, and a collection container having a plurality of ports for collecting patient media. A clinician has an ability to increase or decrease vacuum levels without increasing or decreasing flowrate.

In accordance with another embodiment, a portable suction pump for aspirating secretions from a patient's artificial airway is provided. The portable suction pump includes a vacuum tube extending from the patient through a peristaltic pump head, the vacuum tube communicating with a vacuum sensor via a tee-fitting conduit, a processor for receiving vacuum sensor data from the vacuum sensor to determine if a proportional valve needs to be activated to allow air into the vacuum tube to regulate the vacuum levels, and a collection container for collecting patient media. A clinician has an ability to increase or decrease the vacuum levels without increasing or decreasing flowrate.

In accordance with yet another embodiment, a method for aspirating secretions from a patient's artificial airway is provided. The method includes extending a vacuum tube from the patient through a peristaltic pump head, enabling communication between the vacuum tube and a vacuum sensor via a tee-fitting conduit, receiving, via a processor, vacuum sensor data to determine if a proportional valve needs to be activated to allow air into the vacuum tube to limit the vacuum levels, and collecting patient media by a collection container. A clinician has an ability to increase or decrease the vacuum levels without increasing or decreasing flowrate.

It should be noted that the exemplary embodiments are described with reference to different subject-matters. In particular, some embodiments are described with reference to method type claims whereas other embodiments have been described with reference to apparatus type claims. However, a person skilled in the art will gather from the above and the following description that, unless otherwise notified, in addition to any combination of features belonging to one type of subject-matter, also any combination between features relating to different subject-matters, in particular, between features of the method type claims, and features of the apparatus type claims, is considered as to be described within this document.

These and other features and advantages will become apparent from the following detailed description of illustrative embodiments thereof, which is to be read in connection with the accompanying drawings.

### BRIEF DESCRIPTION OF THE DRAWINGS

The invention will provide details in the following description of preferred embodiments with reference to the following figures wherein:

FIG. 1 illustrates an exemplary portable microprocessor-controlled peristaltic suction apparatus, in accordance with an embodiment of the present invention;

FIG. 2A is a perspective top view of the portable microprocessor-controlled peristaltic suction apparatus of FIG. 1, in accordance with an embodiment of the present invention;

FIG. 2B is a perspective bottom view of the portable microprocessor-controlled peristaltic suction apparatus of FIG. 1, in accordance with an embodiment of the present invention;

FIGS. 3A-3B are perspective top views of the interior components of the portable microprocessor-controlled peristaltic suction apparatus when the cover display assembly is removed, in accordance with an embodiment of the present invention;

FIG. 4 illustrates a block/flow diagram of exemplary logic of metering air into a vacuum tube to limit vacuum, in accordance with an embodiment of the present invention;

FIG. 5 illustrates a block/flow diagram of an exemplary vacuum tube and collection container, in accordance with an embodiment of the present invention;

FIG. 6 illustrates an exemplary collection bag, in accordance with another embodiment of the present invention;

FIG. 7 illustrates a block/flow diagram of an exemplary message and signal control system, in accordance with an embodiment of the present invention;

FIG. 8 illustrates the portable microprocessor-controlled peristaltic suction apparatus including wireless capabilities, a remote control activation function, and a flush feature, in accordance with an embodiment of the present invention; and

FIG. 9 illustrates the flip top of the pump head opened to expose the pump tubing going through the pump head, in accordance with an embodiment of the present invention.

Throughout the drawings, same or similar reference numerals represent the same or similar elements.

### DETAILED DESCRIPTION

Embodiments in accordance with the present invention provide for a method of generating sufficient vacuum to effectively remove secretion from a patient's airway while separately controlling the amount of air and oxygen evacuated from the patient's lungs. The exemplary embodiments further manage suction levels for various types of suction procedures and for different levels of viscosity.

There are several portable suction pumps and several suction regulators in hospitals connected to central suction systems, all having one common feature. They all need an air-filled container (e.g., a suction canister) with a rigid wall that does not collapse under vacuum. Some have internal plastic bag type liners but need a special sealed structure to hold them open for receiving fluids. These suction canisters are connected to a vacuum pump or central vacuum source. Inside the canister is an open space of vacuum where liquids settle to the bottom. The canister has tubing that a clinician connects to the suction catheter for suctioning secretions from the patient's breathing tube. When the clinician inserts the catheter into the breathing tube and applies suction, air and liquids from the patient are drawn into the vacuum inside the canister.

While suctioning is standard in the medical industry, suctioning can be associated with numerous and frequent unintended adverse events. These adverse events are evident whether a medical professional is practicing as a bedside clinician or if a researcher is reviewing published articles on suction procedures, standards, guidelines, and cautions. This is especially true in the neonatal population.

The present suction standard utilizes a compressor type pump to generate vacuum. The vacuum levels are controlled by a regulator or relief valve. What is being controlled is the level of vacuum inside the rigid collection container or canister. What is not being controlled is the flow rate at which fluids are extracted and enter the vacuum filled canister. The flow rates during extraction are determined by the internal diameters of the suction tubing and the level of vacuum inside the canister. Therefore, the higher the vacuum inside the canister, the higher the flow rate of fluid extraction. The present standard suction instruments for clearing secretions from a patient breathing tube cannot reduce flow without reducing vacuum or cannot increase vacuum without increasing flow rates. Hospital central vacuum systems cannot increase vacuum without increasing flow rates. Without control of air flow rates independent of vacuum levels, standard suction equipment used to help maintain an open airway for breathing remains deficient. Excessive flow rates can be the cause of collateral damage during standard suction procedures. Excessive flow rates during suctioning can contribute to adverse events by generating negative pressures in the lung, thus causing collapse of the alveoli. Excessive flow rates during suctioning can contribute to adverse events by unnecessarily evacuating excessive amounts of air and oxygen from the lung, thus causing collapse of the alveoli leading to hypoxia and bradycardia.

Unintended adverse events can be mitigated by generating vacuum for certain procedures using low, constant, or intermittent controlled air flow rates. With all that in mind, the exemplary embodiments introduce a peristaltic pump that generates the needed vacuum to perform the evacuation of liquids from the patient without negative effects of excessive air flow rates. The exemplary embodiments of the present invention introduce a suction pump that provides a wide range of vacuum or suction levels to the user with separate independent control over increasing or decreasing the flow rate in either direction. With standard portable suction pumps and hospital suction regulators, when the suction level is increased, the air flow rate increases proportionally. However, the exemplary embodiments of the present invention introduce a portable suction pump where the suction level can be increased without an increase in flow rate, in contrast to conventional systems and methods. In the exemplary embodiments, the flow rate is constant no matter what level of suction is needed, and the flow rate remains the same.

Thus, the exemplary embodiments introduce a peristaltic pump that monitors, regulates, limits, and manages the vacuum for specific medical procedures.

The exemplary embodiments introduce a peristaltic pump that has the patient media extracted from the patient and isolated from the pump, the atmosphere, vacuum regulators, central hospital piping systems and their compressors.

The exemplary embodiments overcome the hazards and adverse effects associated with suctioning patients with endotracheal and tracheostomy tubes. The exemplary embodiments introduce a method of generating sufficient vacuum to effectively remove secretion from a patient's artificial airway while minimizing the evacuation of air and oxygen from the patient's lungs.

The exemplary embodiments introduce an aspiration pump that generates suction levels well within standard medical practice with a fraction of air flow compared to standard suction, thereby reducing lung collapse and adverse events.

The exemplary embodiments introduce an aspiration or suction pump that provides a wide range of vacuum or suction levels by utilizing custom algorithms using data from a vacuum sensor to control an electro-mechanical proportional valve that meters air into the vacuum tube all while maintaining independently controlled flow rates. The flow rate may be changed by the RPM of the pump or by changing the ID of the pump tubing. Flow can be stopped on the dime, reversed direction, at intervals based on custom algorithms for independent control over flow direction, vacuum levels and flow rates.

The exemplary embodiments introduce a suction tube that connects to standard suction catheters and tubes that are inserted into patients' airways, and oral and nasal cavities, with the intent of removing secretions. The suction tube goes through a roller pump and has a collection container on the other end. On the vacuum side of the roller pump is a tee-fitting with a conduit that connects to a vacuum sensor and a proportional valve to meter air into the suction tube as a method of regulating the vacuum level. The vacuum level in the suction tube is regulated by metering air into the suction tube. Vacuum levels in the suction tube may also be regulated by an adjustable spring loaded vacuum relief valve, pump speed, pump stopping or pump reversing.

On the positive pressure side of the roller pump is a collection container (or, alternatively, a collection bag). The collection container has a vent to atmosphere. The vent has a bacteria filter installed to prevent any contaminates from exiting the collection container and entering the atmosphere. The collection container also has a separate conduit that connects to a pressure sensor to signal the user when the canister is full. The conduit to the pressure sensor may also be incorporated into a canister vent port. The collection container may also be configured in a way that the pressure conduit is isolated from the canister contents by way of a separate bladder to isolate the extracted contents from the pressure conduit.

The exemplary embodiments further introduce a portable suction system that isolates extracted contents and provides safety features that prevent human error from allowing a breach where isolated media contacts the atmosphere, pump, regulator, or central vacuum systems. While standard vacuum systems have bacteria filters to protect the pump, regulator, or central vacuum system, if a filter is not installed, the vacuum works fine. When these systems are used without a bacteria filter (human error) the damage is done and almost impossible to correct. The exemplary embodiments provide for a microprocessor-controlled pump with vacuum and pressure sensors using logic that prevents the suction device from working unless all connections and filters are in place.

The exemplary embodiments can further provide the user with audio feedback while performing a suction procedure. During a suction procedure, the clinician is focused on the patient and the catheter while the suction controls and gauge are out of view. The clinician does not know how much vacuum is needed to extract the secretions. For instance, did the vacuum reach its peak? Did the vacuum need to be so high? The exemplary embodiments thus can provide an audio signal to the clinician during the procedure to alert or notify the clinician when the vacuum reaches its peak during suction. This is an indication that the vacuum level is not high enough to clear the secretions.

The exemplary embodiments further collect and store historical vacuum data that the clinician can access for analysis associated with outcomes.

It is to be understood that the present invention will be described in terms of a given illustrative architecture; however, other architectures, structures, substrate materials and process features and steps/blocks can be varied within the scope of the present invention. It should be noted that certain features cannot be shown in all figures for the sake of clarity. This is not intended to be interpreted as a limitation of any particular embodiment, or illustration, or scope of the claims.

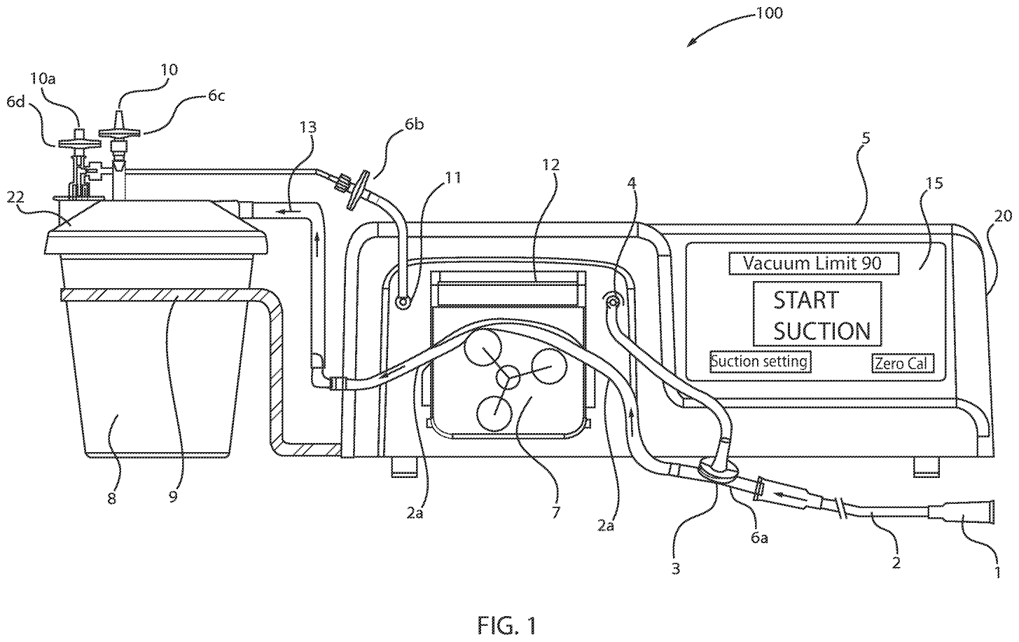

FIG. 1 illustrates an exemplary portable microprocessor-controlled peristaltic suction apparatus 100, in accordance with an embodiment of the present invention.

A peristaltic pump is a type of positive displacement pump used for pumping a variety of fluids. A peristaltic pump can also be referred to as a roller pump. The fluid is contained within a flexible tube fitted inside a circular pump casing (though linear peristaltic pumps have also been made). A rotor with a number of “rollers,” “shoes,” “wipers,” or “lobes” attached to the external circumference of the rotor compresses the flexible tube. As the rotor turns, the part of the tube under compression is pinched closed (or “occludes”), thus forcing the fluid to be pumped or moved through the tube. Additionally, as the tube opens to its natural state after the passing of the cam (“restitution” or “resilience”) fluid flow is induced to the tube. This process is called peristalsis and is used in many biological systems such as the gastrointestinal tract. Usually, there will be three or more rollers, or wipers, occluding the tube, trapping between them a body of fluid. The body of fluid is then transported, under positive pressure, toward the recipient. Peristaltic pumps may run continuously, or they may be indexed through partial revolutions to deliver smaller amounts of fluid.

Peristaltic pumps are used to pump clean/sterile or aggressive fluids without exposing those fluids to contamination from exposed pump components. Some common applications include pumping IV fluids through an infusion device, aggressive chemicals, high solids slurries and other materials where isolation of the product from the environment, and the environment from the product, are beneficial. Peristaltic pumps are also used in heart-lung machines to circulate blood during a bypass surgery, and in hemodialysis systems, as the pump does not cause significant hemolysis.

Peristaltic pumps are further used in a wide variety of industrial applications, especially agriculture as they are well suited for common agricultural chemicals. Their unique design makes roller pumps especially suited to pumping abrasives and viscous fluids.

Standard vacuum pumps have a vacuum gauge with a way to adjust, limit or regulate the level of suction. This is also true for suction regulators that are attached to wall or central suction often used in hospitals. Standard peristaltic pumps operate without regard to vacuum levels other than what is needed to transfer the type of fluid from point A to point B. Therefore, special design features are needed to have a peristaltic pump function as a suction device for removing fluids from a patient's body and adhere to clinical standards and procedures. These design features include but are not limited to pump speed, pump start and stopping venting air into the vacuum tube with a fixed orifice, manually adjustable spring loaded vacuum relief valve, manual needle valve or electronic proportional valve.

The exemplary embodiments of the present invention relate to features needed to utilize the suction side of peristaltic pumps to benefit patients exposed to various suction procedures during medical treatment and recovery. Due to the way negative pressures are generated by the peristaltic pump, it is not sufficient to utilize the negative pressures for medical procedures without design features that provide for safety and effectiveness.

Specifically, the exemplary embodiments of the present invention specify the design features of the conduit through the peristaltic pump head to the collection container. The pump head has a flip top that provides easy access to install the pump tube. There are other style pump heads that provide quick access for installing different size pump tubes. This conduit begins at the connection to the patient's tube and ends at the collection container. Conventional systems and methods focus on quantifying the level of vacuum on the intake side of the pump and the amount of pressure on the output side. All sorts of systems and methods are presented for ways to have this vacuum/pressure level of control without the use of an ancillary orifice to a sensor to avoid the possibility of air or contamination of the liquid. These constraints require unique features and are completely understandable when the mission is circulating blood from one organ to another, eliminating the possibility of contamination, or exposure.

However the exemplary embodiments of the present invention involve extracting fluids from the patient with the vacuum side of the peristaltic pump and the pressure side is used for collection. Therefore, having air enter the vacuum side does not need to be avoided but to the contrary, is used as the way to accurately control and limit vacuum levels.

The exemplary embodiments of the present invention use an electronic proportional valve controlled by a microprocessor that meters air into the vacuum tube based upon feedback from the vacuum sensor. The control panel on the touch screen allows the user to select a vacuum limit and the microprocessor meters air into the vacuum tube via the electronic proportional valve based upon feedback from the vacuum sensor to achieve the vacuum limit selected by the user.

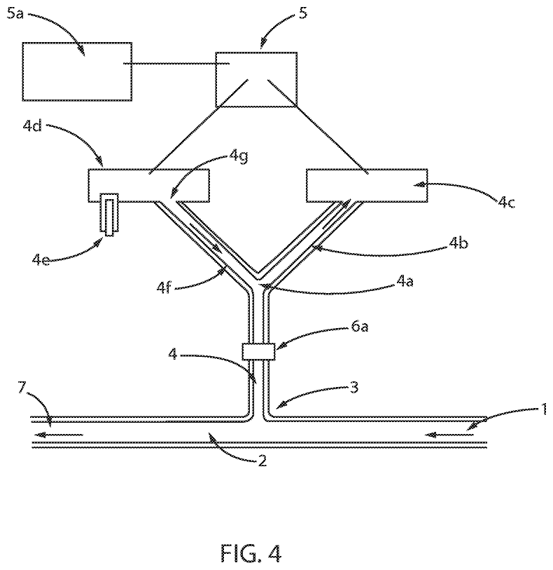

Referring to FIG. 1, the suction tube 2 (or vacuum tube 2) extends from the patient 1 through the peristaltic pump head 7 to the collection canister or container 8. The section of the vacuum tube 2 between the patient 1 and the pump head 7 is the vacuum section. In this section of the vacuum tube is installed a tee-fitting conduit 3. The tee-fitting conduit 3 has an in-line filter 6a. The tee-fitting conduit 3 extends to a vacuum sensor port that connects to the internal vacuum sensor 4c (FIG. 4) for communication with the microprocessor 5. The microprocessor 5 controls a proportional valve 4d (FIG. 4) employed to regulate the vacuum levels the user selects with the touch screen 15. The suction tube 2 extending from the patient 1 connects to the pump tube 2a and passes into and through the pump head 7. The flip top 12 opens the pump head 7 to allow the suction tube 2 to be installed. Once installed, the top is closed with the suction tube 2 inside the pump head 7. The suction tube 2 can expand and contract. The suction tube 2 can expand to enable removal of secretion from a patient's airway and contract to occlude application of vacuum. The contraction occurs within the pump head 7.

Patient media is pumped into collection canister or container 8. The collection canister 8 is under positive pressure and has three ports communicating through the lid 22. A first port 13 is where the patient media is pumped into the canister 8. The second and third ports 10, 10a provide air vents to the atmosphere. The air vent ports incorporate viral filters 6c, 6d to protect the atmosphere from the patient media. When the canister 8 is full, the internal liquid check valve closes and the primary vent 10 closes. The third port 10a is a secondary vent that remains active when the canister 8 is full. It incorporates the tee-fitting conduit 3 with a tube and filter 6b that extends to the pressure sensor port 11. It connects to the internal pressure sensor for communication with the microprocessor 5. When the canister 8 is full, the microprocessor 5 turns the pump off. Therefore, the microprocessor 5 monitors the collection side (positive pressure) and monitors the vacuum side (negative pressure). A bracket 9 can be employed to support the canister 8 with respect to the pump head 7. It is further noted that air is drawn into the vacuum side, as opposed to the delivery system, in contrast to conventional systems.

The portable microprocessor-controlled peristaltic suction apparatus 100 includes a cover-display assembly 20. The cover-display assembly 20 has a display screen 15. A microprocessor 5 is in communication with the control panel 5a (FIG. 4) of the display screen 15. Further, the tee-fitting conduit 3 in the suction tube 2 provides a separate conduit 4. The conduit 4 goes through the bacteria filter 6a intended to block all but air flow and sound pressure.

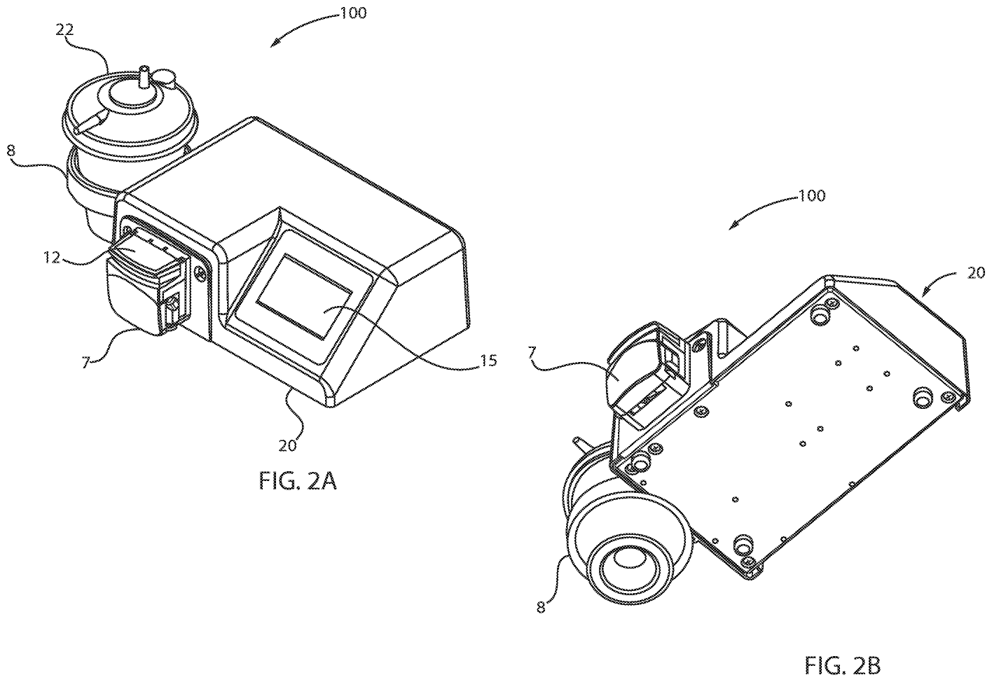

FIG. 2A is a perspective top view of the portable microprocessor-controlled peristaltic suction apparatus, in accordance with an embodiment of the present invention.

The portable microprocessor-controlled peristaltic suction apparatus 100 includes a cover-display assembly 20. The cover-display assembly 20 has a display screen 15, as well as a pump head 7. The pump head 7 includes a flip top 12. The flip top 12 is shown in a closed configuration or closed state. Attached to the cover-display assembly 20 is the collection canister 8 and the bottom portion of the cover-display assembly 20.

FIG. 2B is a perspective bottom view of the portable microprocessor-controlled peristaltic suction apparatus, in accordance with an embodiment of the present invention.

The bottom view of the portable microprocessor-controlled peristaltic suction apparatus 100 better illustrates the collection canister 8, as well as the pump head 7.

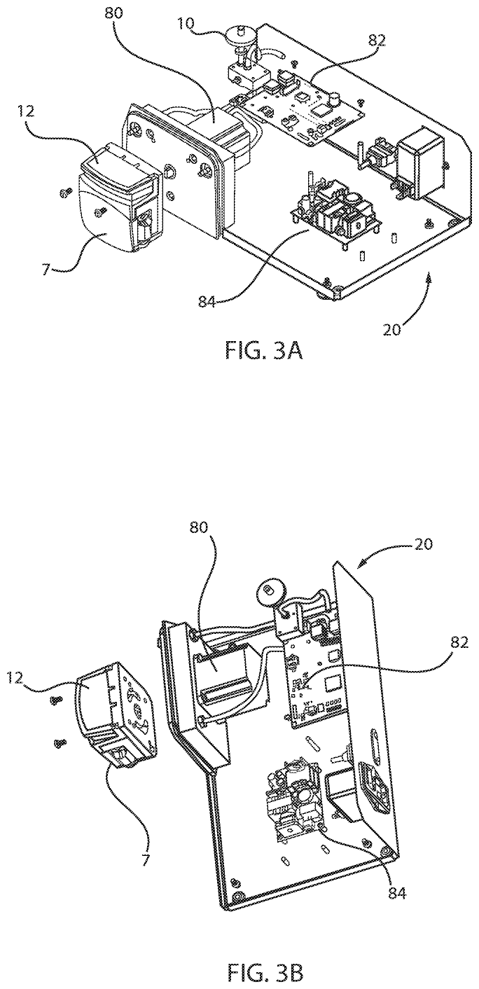

FIGS. 3A-3B are perspective top views of the interior components of the portable microprocessor-controlled peristaltic suction apparatus when the cover display assembly is removed, in accordance with an embodiment of the present invention.

The interior components of the portable microprocessor-controlled peristaltic suction apparatus 100 include at least a motor 80, a sensor control assembly 82, and a power supply 84. The motor 80, the sensor control assembly 82, and the power supply 84 are positioned within the cover-display assembly 20. The pump head 7 with the flip top 12 are positioned or attached to an exterior of the cover-display assembly 20. The positioning of the second port 10 is also shown with respect to the sensor control assembly 82.

FIG. 4 illustrates a block/flow diagram of exemplary logic of metering air into a vacuum tube to limit vacuum, in accordance with an embodiment of the present invention.

FIG. 4 further illustrates how vacuum is controlled or how suction is limited. One skilled in the art is aware that on the vacuum side of the pump conduit, vacuum keeps building up unless fluid is entering the conduit. When the conduit is occluded, the vacuum level keeps increasing with each rotation of the roller pump. The only thing that eventually limits the vacuum level is the collapse of the vacuum tube. Suction pumps used for medical procedures require vacuum limits. If a catheter or suction tube is inside the patient and becomes occluded with soft tissue, the high vacuum can cause injury to the tissue. Therefore, it is important to provide a way to manage the suction level for the type of suction procedure being performed and for the different levels of liquid viscosity.

The user selects the level of suction allowed for the procedure on a control panel 5a controlled by the microprocessor 5. The pump rotation speed is preset, constant, ON or OFF. The vacuum limit set by the user is sent to the microprocessor 5. The microprocessor 5 monitors the collection side (positive pressure) and monitors the vacuum side (negative pressure). Fluid flow from the patient 1 enters the suction tube 2. A tee-fitting conduit 3 in the suction tube 2 provides a separate conduit 4. The conduit 4 goes through a bacteria filter 6a intended to block all but air flow and sound pressure. The separate conduit connects to a wye-fitting 4a where one conduit 4a connects to a vacuum sensor 4c and the other conduit 4f connects to a proportional valve 4d that is either closed or open.

The valve has two ports. One that can be opened to atmospheric air 4e and the other conduit 4g allows metered air to flow into the suction tube 2. The vacuum sensor 4c and the valve 4d connect to the microprocessor 5. When the vacuum level in the suction tube 2 reaches the set limit, the microprocessor 5 instructs the valve 4d to open and allow air from the atmosphere 4e to enter the suction tube 2, thereby controlling the vacuum level.

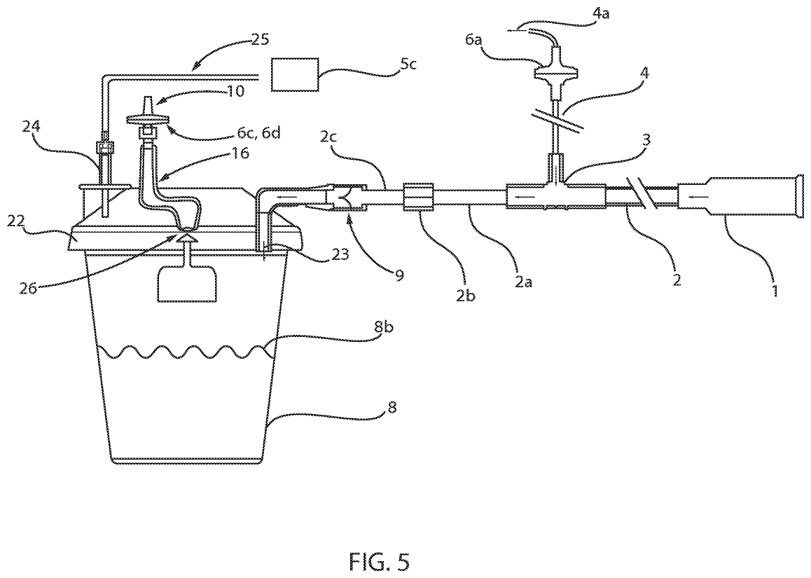

FIG. 5 illustrates a block/flow diagram of an exemplary vacuum tube and collection container, in accordance with an embodiment of the present invention.

FIG. 5 is a schematic of the suction system components that are exposed to and contain the media suctioned from the patient. This system of components isolate such media from the work area and allow for safe and easy disposal. The suction tube from the patient 1 connects to suction tube 2 with tee-fitting conduit 3 and separate lumen (or conduit) 4 leads to bacterial filter 6a. The bacteria filter 6a has a hydrophobic type membrane which allows air to pass through but blocks liquid from passing through. Liquid can pass through hydrophobic membrane material, but it needs a certain amount of pressure. In this system of components, there is not enough pressure for liquid to pass through the hydrophobic membrane. Therefore, bacteria filter 6a effectively isolates the liquid media suctioned from the workplace. Bacterial filter 6a allows sound pressure to reach the vacuum sensor and allows air flow from the microprocessor-controlled electromechanical valve.

Peristaltic pump tubing 2a has certain properties required for working inside the pump head 7. The pump tubing 2a is often constructed of silicone having durable resilient properties. Upon exiting the peristaltic pump head 7 is a section of collection tubing 2c that may utilize a one-way check valve 9. The check valve 9 is useful in the event the user wants to replace the collection canister or container. The check valve 9 prevents liquid from leaking into the work area during handling and disposal.

The suctioned liquid enters the collection canister 8 through port 23. The collection canister 8 is watertight as indicated by weld 8b. The collection canister 8 can have at least one hole or member for holding the canister in an upright position. The collection canister 8 has a second port 24 that has a separate conduit for sending sound pressure to a pressure sensor 5c inside the unit. The conduit from port 24 leads to bacterial filters 6c, 6d. The bacteria filters 6c, 6d have a hydrophobic type membrane, which allows sound pressure to pass through conduit 11 (FIG. 1) or conduit 25 to the pressure sensor 5c. The collection canister 8 has a third port 16 to 10 to provide a vent for air from the collection canister 8. Port 10 has a bacterial filter 6c that has a hydrophobic type membrane which allows air to pass through but blocks liquid from passing through.

When patient media reaches the air vent, it closes off, and pressure in the collection canister 8 increases. The pressure sensor 5c senses the increase in pressure and at a predetermined pressure level a notification is sounded, a message (collection canister 8 is full) is displayed and the pump head 7 turns off. In the instant case, the canister 8 has liquid reaching a liquid level 8b, which is well below the float valve, which has a liquid level shutoff 26 extending within the confines of the lid 22 of the canister 8.

Additionally, an air pressure port 24 extends into the lid 22 of the canister 8. The air pressure port 24 provides an air vent to the atmosphere. The air pressure port 24 protects the atmosphere from the patient media. The air pressure port 24 includes a conduit 25 to the pressure sensor 5c.

It is noted that the tubes that connect to the pressure port and the vacuum sensor port are sized so that they cannot be connected to the wrong port. In other words, the ports are sized to only accept the right sized or appropriate sized tube. In another embodiment, the tubes can be color-coded such that a first color tube is connected to one port and a second color tube is connected to another port. The terms “color-coded” and “color” as used herein mean any visual way of distinguishing different sections along the tube body. This includes differentiation based on color and/or luminescence such as phosphorescence or fluorescence. For example, sections of the tube or the entire tube can be of distinct colors such as, e.g., red or green or yellow. Sections can also be luminescent sections, either alone instead of a color, or in addition to color-coded sections. Each color or color-coded section corresponds with one or more distinct ports.

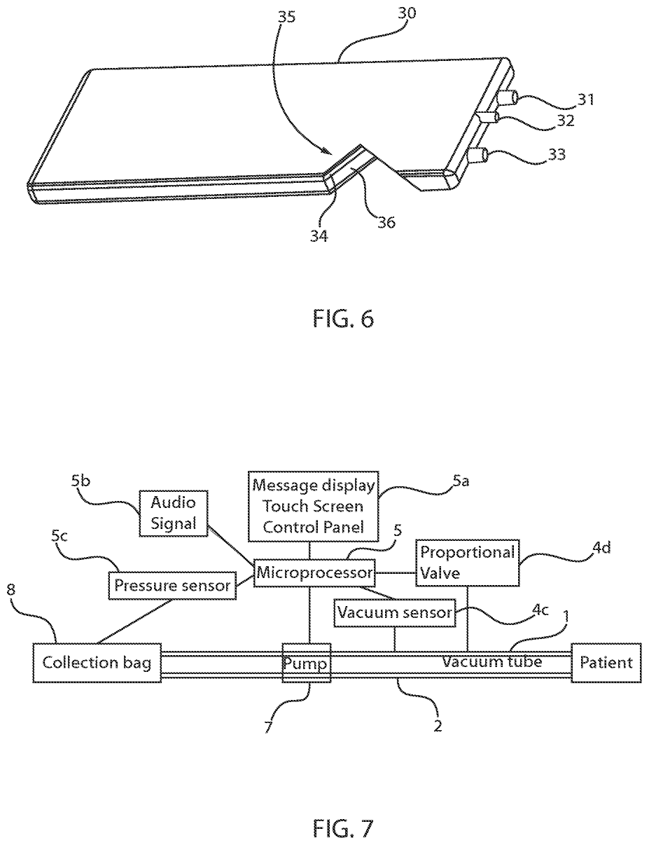

FIG. 6 illustrates an exemplary collection bag, in accordance with another embodiment of the present invention. The collection bag may be of a material having elastic qualities.

FIG. 6 depicts a safety measure of always having a patient conduit to the pressure sensor 5c. This is accomplished with the collection bag 30 having two chambers. The first chamber 33 is for aspirated liquids. The second chamber 32 is sealed and isolated from the first chamber 33 and has air trapped (with or without the aid of compressible elastic spongy material) and is connected to the pressure sensor 5c. The second chamber 32 may be inside or adjacent to the first collection chamber 33. When the first chamber 33 becomes full and causes the air vent 31 to a hydrophobic filter to close, the increased pressure is applied to the second chamber 32 connected to the pressure sensor 5c. A predetermined pressure level in the second chamber 32 activates a notification sound, a message (collection bag full) is displayed and the head pump 7 turns off. This embodiment has the advantage of completely isolating the pressure conduit from aspirated patient media. A cutout or cut away of the collection bag 30 illustrates the air pressure channel 34 and the collection bag channel 36 of the dual channel collection bag 35.

FIG. 7 illustrates a block/flow diagram of an exemplary message and signal control system, in accordance with an embodiment of the present invention.

FIG. 7 is a schematic that describes the systems function and logic. Vacuum tube 1 is the suction tube from the patient. The vacuum sensor 4c has a conduit to the suction tube 1. Data from the vacuum sensor 4c goes to the microprocessor 5. The microprocessor 5 directs a digital display of the vacuum level on the control panel 5a of display screen 15. Vacuum sensor data to the microprocessor 5 determines if the proportional valve 4d needs to be activated to allow air into the suction tube 1 to limit the suction level. Vacuum sensor data to the microprocessor 5 determines if the vacuum level limit in the suction tube 1 has been reached and if so activates an audio signal 5b. The microprocessor 5 logs the number of times the unit was used, the level of suction set and the average suction level used. Vacuum tube 1 goes into the peristaltic pump head 7 and into the collection canister 8. The collection canister 8 has a conduit to the pressure sensor 5c. The pressure sensor 5c communicates with the microprocessor 5. When the pump head 7 is turned on, the microprocessor 5 reads the positive pressure signal from the pressure sensor 5c. If there is no pressure, the pump head 7 turns off and displays an error code, such as, e.g., Check Collection Canister Connections. When the pump head 7 is turned on, the microprocessor 5 reads the vacuum level in the suction tube 1. If there is no vacuum, the pump head 7 turns off and displays an error code, such as, e.g., Check Suction Tube Connections. The microprocessor 5 turns the pump on for continuous vacuum, intermittent vacuum, timed on intervals, timed off intervals, and reverse (positive pressure) intervals.

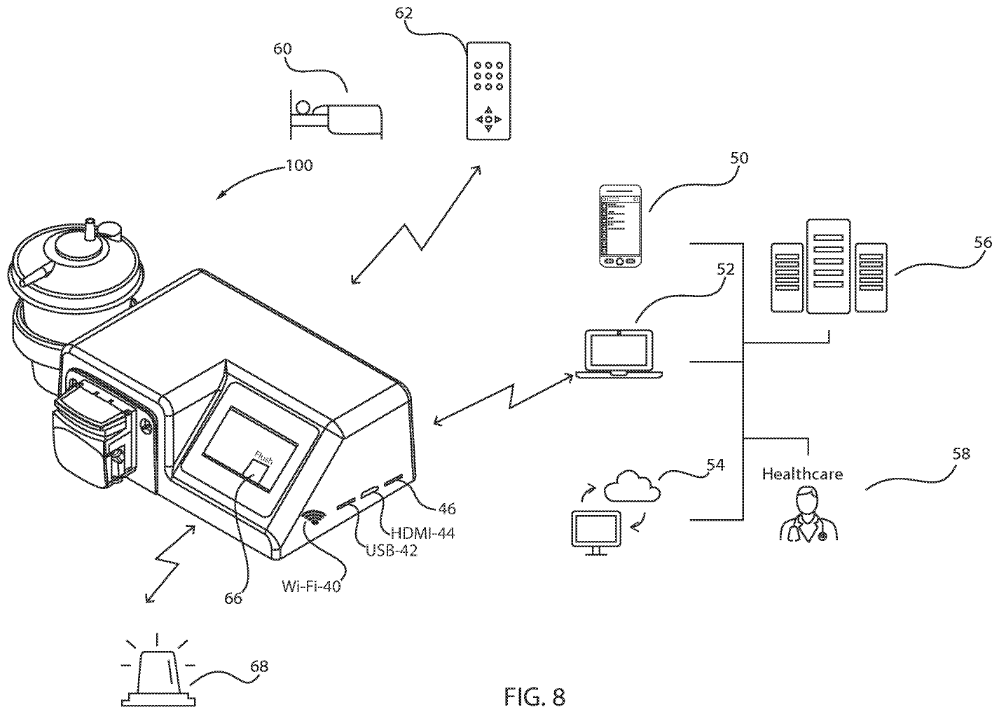

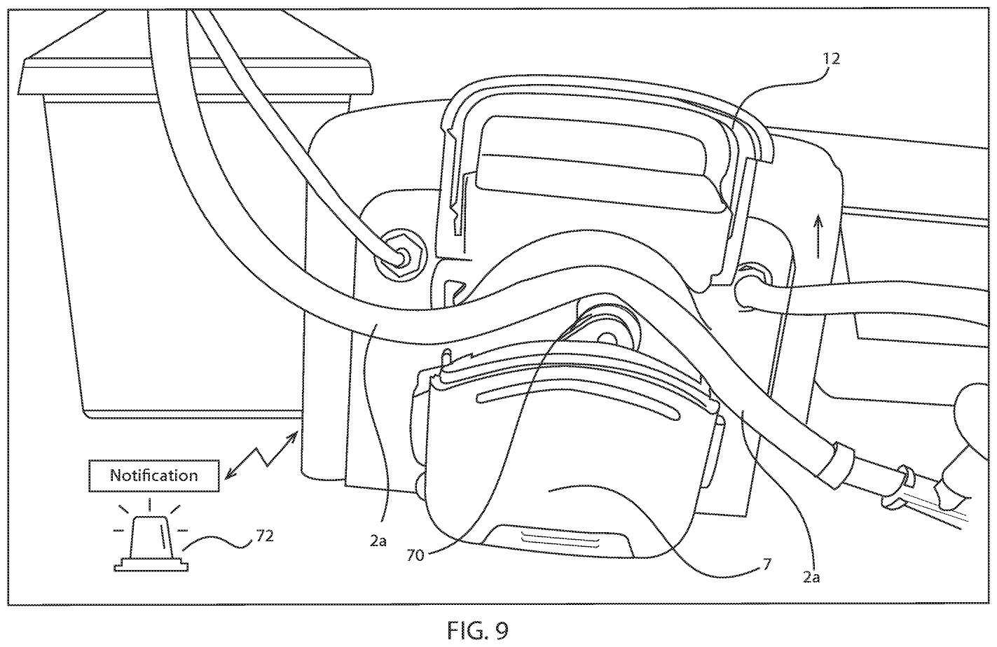

FIG. 8 illustrates the portable microprocessor-controlled peristaltic suction apparatus including wireless capabilities, a remote control activation function, and a flush feature, in accordance with an embodiment of the present invention.

The portable microprocessor-controlled peristaltic suction apparatus 100 can also include wireless capabilities. The apparatus 100 can include a WiFi connection 40, a USB slot 42, an HDMI slot 44, and a memory slot 46. One skilled in the art can contemplate any other types of connections to enable wireless or wired communications. Thus, the apparatus 100 can include a plurality of ports for communicating with one or more peripheral devices and/or one or more other devices. For example, an HDMI port, a DVI port, a plurality of USB ports, an Ethernet port, a WiFi transceiver, and/or the like can be provided. In the instant case, e.g., the apparatus 100 can communicate with a mobile device 50, a laptop 52 or to any other computing device via, e.g., cloud 54. The collected data can be stored in one or more servers 56 and provided to healthcare professionals 58.

In another exemplary embodiment, a remote control 62 can be provided to a patient 60 in bed. The patient 60 in bed can use the remote control 62 to operate the apparatus 100. The patient 60 can activate, deactivate any functions of the apparatus 100 remotely from his/her bed. As a result, the patient 60 does not need to call a nurse or caregiver to assist with use of apparatus 100. In addition, the remote control 62 permits the patient 60 to control or operate the apparatus 100 remotely without the need to touch any buttons or other parts of the apparatus 100, which could also reduce the likelihood of transmitting infection between the nurse or caregiver and the patient 60.

The remote control 62 may include, for example, control buttons, a keypad, a pen-based or stylus-based input, voice recognition input, touch screen input, bar code scanner or other suitable input devices commonly available.

In another exemplary embodiment, a flush button 66 can be provided on the display screen 15. The flush button 66 allows for a user to override a vacuum limit or vacuum level. The flush vacuum level can be set to, e.g., 5 seconds of vacuum application. A notification 68 can be transmitted when the vacuum level is reached (e.g., at the 5 second mark). However, application of the vacuum need not terminate when the vacuum level has been reached. The application of the vacuum can continue past this point, that is, past 5 seconds in the instant example. The vacuum is applied to clean the suction tube 2 which extends through the pump head 7. In one instance, activation of the flush button 66 triggers the suction tube 2 to be compressed to occlude application of vacuum, the compression of the suction tube 2 occurring within the pump head 7.

Moreover, apparatus 100 may include an acoustic transmitter for emitting a warning or alarm signal 68. In the alternative or in addition, the apparatus 100 may have a visual indicator. A visual indicator can be less disturbing to patients and hospital staff than an acoustic indicator and is especially useful for indicating status and minor alarms. A visual indicator is also optimal during periods of rest, for instance at night, when the indicator remains effective even when ambient lighting has been lowered or switched off.

The indicator of the device can generally be used to provide a status, warning or alarm condition 68 associated with: (i) a patient (e.g. physiological parameters of a patient, such as pulse rate, blood pressure, body temperature or respiration rate, or an environmental condition of the patient, such as adjacent moisture or humidity levels), (ii) therapy, therapy status being applied or delivered to the patient, or the patient's response to the delivered therapy (e.g. therapy duration, therapy rate, etc.), and/or (iii) the status of the apparatus 100 performing and/or delivering the therapy to the patient (e.g. any detected device failures or defects, disconnection of device components, low or no power warnings, electrical surge or high temperature warnings, other device environmental warnings, device maintenance warnings, vacuum limits/levels, suction tube operation, etc.).

FIG. 9 illustrates the flip top of the pump head opened to expose the pump tubing going through the pump head, in accordance with an embodiment of the present invention.

In FIG. 9, the flip top 12 is opened to expose the suction tube 2a extending through the pump head 7. The suction tube 2a rests on a wheel 70 within the pump head 7. When the flip top 12 is in the open configuration or in the open state, application of vacuum is automatically ceased. This is considered a safety feature. A notification 72 can also be activated when the flip top 12 is in an open state. Any type of acoustic or visual notification can be contemplated by one skilled in the art.

In another exemplary embodiment, the features include receiving, at wireless devices or one or more servers or sending to healthcare professionals, one or more of electronic medical record data for the patient, profile data for the patient, care plan data for the patient, or social networking data for the patient. The features may include calculating at least one of an event for the patient, updated profile data for the patient, or updated care plan data for the patient. The features may include transmitting patient-generated data from the wireless device to the one or more servers or healthcare professionals. The features may include receiving, at the wireless devices or one or more servers, outcome data indicating an outcome of a health event of the user, analyzing the outcome data, and transmitting at least a portion of the outcome data or one or more results of analyzing the outcome data. The outcome data can relate to vacuum data. The vacuum data can include, e.g., how many times the apparatus 100 was used in 24 hours, in a week, in a month, etc., what were the average vacuum levels, how many times the flush button was activated, at what times of day was the flush button activated, how many times a patient remotely accessed the apparatus 100, what functions did the patient access remotely, etc. Any data contemplated by one skilled in the art and related to vacuum information can be collected and stored.

Therefore, in summary, a portable suction pump 100 is presented for aspirating secretions from a patient's artificial airway that provides the clinician with the ability to increase vacuum levels without increasing flowrate. The portable suction pump uses a roller pump to generate flow and vacuum, and has a separate single conduit positioned into the suction tube to control vacuum levels. The separate conduit communicates with a vacuum sensor and a proportional valve that communicates with a microprocessor to control vacuum levels in the suction tube. The portable suction pump uses a roller pump to generate flow and vacuum where the flow rate is controlled by the speed of pump rotation and the internal diameter of the pump tube. The vacuum levels in the suction tube are controlled by, e.g., an algorithm or software code in the microprocessor to instruct the proportional valve to open or close depending on the dynamic vacuum levels in the suction tube during a suction procedure.

In further exemplary embodiments, the portable suction pump 100 uses a roller pump to generate flow and vacuum with a collection container where the liquid is collected and the air vents to the atmosphere. The collection container has a separate conduit that connects to a pressure sensor that communicates with the microprocessor to signal the user when the collection container is full. The collection container can have a vent that filters the air before it enters the room. The collection container can have at least one hole or member to support the canister in an upright position. The collection container can have a vent that has a float valve and secondary orifice to warn the user that the collection container is full.

In further exemplary embodiments, alternatively, a collection bag is presented with an internal pouch that connects to the pressure sensor. The collection bag can have two sections. One section for collection of fluids and the other section for monitoring the pressure in the collection bag. The portable suction pump can use a roller pump to generate flow and vacuum with a microprocessor that monitors vacuum and pressure in the suction circuit and the suction circuit has a one-way check valve. The portable suction pump can use a roller pump to generate flow and vacuum with a microprocessor that monitors vacuum and pressure in the suction circuit and shuts down if the suction circuit is not complete.

The portable suction pump 100 can use a roller pump to generate flow and vacuum with a microprocessor that monitors vacuum and pressure in the suction circuit and provides an audio signal when the vacuum level reaches the set or predetermined limit or threshold. The portable suction pump 100 can use a roller pump to generate flow and vacuum with a microprocessor that monitors vacuum and pressure in the suction circuit, and logs suction data for retrieval and analysis. The portable suction pump 100 can use a roller pump to generate flow and vacuum with a microprocessor that monitors vacuum and pressure in the suction circuit and opens the circuit to atmosphere when a zero-calibration button is activated. The portable suction pump 100 can use a roller pump to generate flow and vacuum with a microprocessor that monitors vacuum and pressure in the suction circuit and has a “flush” option or flush button (FIG. 8) that provides maximum suction for approximately 5 seconds to help clear debris from the suction circuit.

In another embodiment consistent with the present invention, medical data mining offers the potential to enhance workflow and diagnostic accuracy through objective data-driven analytics, which can be categorized in accordance with specific variables relating to the individual exam, patient, provider, and technology being utilized.

Workflow derived data provides an objective tool for the program to evaluate cause and effect and also provides an objective method of assessing technology performance. In addition, data mining can provide valuable insight as to the relationship between workflow and quality, with the goal and objective of any workflow optimization strategy being the simultaneous improvement of productivity and quality.

In the exemplary embodiments, data mining can relate to medical data mining, and more specifically, to at least historical vacuum/suction level data. Data mining synergistically improves both productivity and quality, through the combined analysis of examination complexity, interpretation accuracy, and interpretation times, specific to each individual medical procedure.

The medical data mining of the exemplary embodiments of the present invention can be used in a number of applications within medical imaging, including the creation of best practice guidelines (i.e., evidence-based medicine), improving patient safety (e.g., radiation and contrast optimization), and assessment of quality deliverables among various stakeholder groups. These data mining applications share the common goal of enhancing clinical outcomes, which is the most important goal in medicine. The data mining applications can be used in accordance with artificial intelligence or machine learning techniques.

Machine learning may incorporate predictive model algorithms to execute predictive analytical operations. Learning may be supervised or unsupervised. In general, a predictive model analyzes historical data to identify patterns in the medical data. The patterns identified may include relationships between various events, characteristics, or other attributes of the medical data being analyzed. Modeling of such patterns may provide a predictive model whereby predictions may be made. Development of predictive models may employ mathematical or statistical modeling techniques such as curve fitting, smoothing, and regression analysis to fit or train the data. Such techniques may be used to model the distribution and relationships of the variables, e.g., how one or more events, characteristics, or circumstances (which may be referred to as “independent variables” or “predictor variables”) relate to an event or outcome (which may be referred to as a “dependent variable” or “response”). Such predictive models can be used with respect to the peristaltic pump 100, and in particular, pertaining to vacuum level data/information.

For example, a dataset including observed medical data (e.g., vacuum levels) may be input into a modeling process for mapping of the variables within the medical data. The mapped medical data may be used to develop a predictive model. The machine learning process may also include utilizing the predictive model to make predictions regarding a specified outcome that is a dependent variable with respect to the predictive model. The machine may then be provided an input of one or more observed predictor variables upon which the output or response is requested. By executing the machine learning algorithm utilizing the input, the requested response may be generated and outputted. Thus, based on the presence or occurrence of a known predictor variable, the machine learning algorithm may be used to predict a related future event or the probability of the future event, such events related to, e.g., vacuum levels output by the peristaltic pump 100.

The present disclosure describes various modules, which may also be referred to as sub-modules, generators, engines, systems, subsystems, components, units, and the like. Such modules may include functionally related hardware, instructions, firmware, or software. Modules may include physical or logical grouping of functionally related applications, services, resources, assets, systems, programs, databases, or the like. Modules or hardware storing instructions or configured to execute functionalities of the modules may be physically located in one or more physical locations. For example, modules may be distributed across one or more networks, systems, devices, or combination thereof. It will be appreciated that the various functionalities of these features may be modular, distributed, and/or integrated over one or more physical devices.

While there have been shown, described and pointed out fundamental novel features of the present principles, it will be understood that various omissions, substitutions and changes in the form and details of the methods described and devices illustrated, and in their operation, may be made by those skilled in the art without departing from the spirit of the same. For example, it is expressly intended that all combinations of those elements and/or method steps which perform substantially the same function in substantially the same way to achieve the same results are within the scope of the present principles. Moreover, it should be recognized that structures and/or elements and/or method steps shown and/or described in connection with any disclosed form or implementation of the present principles may be incorporated in any other disclosed, described or suggested form or implementation as a general matter of design choice. It is the intention, therefore, to be limited only as indicated by the scope of the claims appended hereto.

It should also be understood that the example embodiments disclosed and taught herein are susceptible to numerous and various modifications and alternative forms. Thus, the use of a singular term, such as, but not limited to, “a” and the like, is not intended as limiting of the number of items. Furthermore, the naming conventions for the various components, functions, parameters, thresholds, and other elements used herein are provided as examples, and can be given a different name or label. The use of the term “or” is not limited to exclusive “or” but can also mean “and/or”.

Having described preferred embodiments, which serve to illustrate various concepts, structures and techniques that are the subject of this patent, it will now become apparent to those of ordinary skill in the art that other embodiments incorporating these concepts, structures and techniques may be used. Additionally, elements of different embodiments described herein may be combined to form other embodiments not specifically set forth above.

Accordingly, it is submitted that that scope of the patent should not be limited to the described embodiments but rather should be limited only by the spirit and scope of the following claims.

1. A portable suction pump for aspirating secretions from a patient's artificial airway, the portable suction pump comprising:
a peristaltic pump head;
a suction tube extending from a patient through the peristaltic pump head, the suction tube having a tee-fitting conduit; and
a collection container having a plurality of ports for collecting patient media,
wherein a clinician has an ability to increase or decrease vacuum levels without increasing or decreasing flowrate.

2. The portable suction pump of claim 1, wherein the tee-fitting conduit extends to a vacuum sensor port connecting to a vacuum sensor in communication with a processor, the processor controlling a proportional valve employed to regulate the vacuum levels.

3. The portable suction pump of claim 2, wherein the tee-fitting conduit connects to a wye-fitting where one conduit connects to the vacuum sensor and the other conduit connects to the proportional valve, the proportional valve having two ports.

4. The portable suction pump of claim 3, wherein the first port is opened to atmospheric air and the second port allows metered air to flow into the suction tube.

5. The portable suction pump of claim 4, wherein, when a vacuum level in the suction tube reaches a predetermined threshold, the processor instructs the proportional valve to open and allow air from the atmosphere to enter the suction tube to control the vacuum levels.

6. The portable suction pump of claim 1, wherein the plurality of ports include a first port of the collection container for receiving the patient media, and second and third ports of the collection container for providing air vents to the atmosphere, the air vents incorporating viral filters to protect the atmosphere from the patient media.

7. The portable suction pump of claim 6, wherein, when the collection container is full, an internal liquid check valve closes and the second port closes.

8. The portable suction pump of claim 6, wherein the third port remains open when the collection container is full.

9. The portable suction pump of claim 6, wherein, when the collection container is full, a processor turns off the portable suction pump.

10. The portable suction pump of claim 1, wherein the collection container has a first chamber and a second chamber, the first chamber collecting the patient media and the second chamber monitoring pressure within the collection container.

11. A portable suction pump for aspirating secretions from a patient's artificial airway, the portable suction pump comprising:
a vacuum tube extending from a patient through a peristaltic pump head, the vacuum tube communicating with a vacuum sensor via a tee-fitting conduit;
a processor for receiving vacuum sensor data from the vacuum sensor to determine if a proportional valve needs to be activated to allow air into the vacuum tube to regulate the vacuum levels; and
a collection container for collecting patient media,
wherein a clinician has an ability to increase or decrease the vacuum levels without increasing or decreasing flowrate.

12. The portable suction pump of claim 11, wherein the tee-fitting conduit connects to a wye-fitting where one conduit connects to the vacuum sensor and the other conduit connects to the proportional valve.

13. The portable suction pump of claim 11, wherein the proportional valve has two ports, the first port opened to atmospheric air and the second port permitted to meter air to flow into the vacuum tube.

14. The portable suction pump of claim 11, wherein the collection container has a plurality of ports including a first port of the collection container for receiving the patient media, and second and third ports of the collection container for providing air vents to the atmosphere, the air vents incorporating viral filters to protect the atmosphere from the patient media.

15. The portable suction pump of claim 14, wherein, when the collection container is full, an internal liquid check valve closes and the second port closes.

16. The portable suction pump of claim 14, wherein the third port remains open when the collection container is full.

17. The portable suction pump of claim 14, wherein, when the collection container is full, the processor turns off the portable suction pump.

18. A method for aspirating secretions from a patient's artificial airway, the method comprising:
extending a vacuum tube from a patient through a peristaltic pump head;
enabling communication between the vacuum tube and a vacuum sensor via a tee-fitting conduit;
receiving, via a processor, vacuum sensor data to determine if a proportional valve needs to be activated to allow air into the vacuum tube to limit the vacuum levels; and
collecting patient media by a collection container,
wherein a clinician has an ability to increase or decrease the vacuum levels without increasing or decreasing flowrate.

19. The method of claim 18, wherein the tee-fitting conduit connects to a wye-fitting where one conduit connects to the vacuum sensor and the other conduit connects to the proportional valve.

20. The method of claim 18, wherein the proportional valve has two ports, the first port opened to atmospheric air and the second port permitted to meter air to flow into the vacuum tube.
